(19) United States
(12) Patent Application Publication
(10) Pub. No.: US 2021/0186879 A1
(43) Pub. Date: Jun. 24, 2021

(54) TARGETED DELIVERY OF HYDROPHILIC DRUGS TO LUNG

(71) Applicant: DOUBLE BOND PHARMACEUTICALS AB, Uppsala (SE)

(72) Inventor: Igor LOKOT, Uppsala (SE)

(21) Appl. No.: 16/076,179

(22) PCT Filed: Mar. 3, 2017
(86) PCT No.: PCT/EP2017/025035

§ 371 Date: Aug. 7, 2018

(30) Foreign Application Priority Data
Mar. 7, 2016 (SE) 1600083-8

(51) Int. Cl. A61K 9/16; A61K 9/00; A61K 31/704; A61K 31/136; A61K 31/475; A61K 31/4745; A61K 31/473; A61K 31/65; A61K 31/496; A61K 31/4164; A61K 31/425; A61K 31/41; A61K 31/7048; A61K 38/12; A61K 31/4409; A61K 31/43; A61K 31/145

(52) U.S. Cl. CPC A61K 9/1611; A61K 31/145; A61K 31/704; A61K 31/136; A61K 31/475; A61K 31/4745; A61K 31/473; A61K 31/65; A61K 31/496; A61K 31/4164; A61K 31/425; A61K 31/41; A61K 31/7048; A61K 38/12; A61K 31/4409; A61K 31/43; A61K 9/0019

(57) ABSTRACT

A pharmaceutical composition in form of an aqueous suspension up to a size of 200 μm comprises or consists of amphiphilic sulfonate and/or sulfate of a hydrophilic cancer drug having a solubility in water or aqueous body fluid of less than 0.1% by weight. Also disclosed are the particles in powderous form, methods for their production and for the production of the suspension, a method of treating cancer, bacterial or fungal infections in lung by administration of the pharmaceutical composition, and a method of designing a composition according to the invention.

### FIELD OF THE INVENTION

The present invention relates to the administration of hydrophilic drugs to specific sites of the human and animal body, in particular to the lung. More specifically the present invention relates to the administration of hydrophilic drugs, in particular anti-cancer drugs, antifungals and antibiotics.

### BACKGROUND OF THE INVENTION

The therapeutic window represents the range of drug dosages by which a disease can be treated efficiently and safely. It ranges from the dosage at which a noticeable therapeutic effect is seen to that at which the therapeutic benefit is neutralized by adverse effects.

The majority of anticancer drugs have a narrow therapeutic window. In addition it is often a tiny fraction of an administered dose that reaches the site to be treated. Upon systemic administration by oral ingestion or intravascular injection, the medication is distributed throughout the body via the circulation resulting in the entire body being affected. Ideally, the medication should be directed exclusively to a desired body site such as an organ or tissue in need of treatment. Such targeted administration would avoid harming the rest of the body. This kind of administration seeks to direct the medication to tissues of interest while avoiding substantial amounts thereof reaching tissues that do not require treatment.

An example of drugs which need to be directed to a specific body site is the anti-cancer drug doxorubicin. It is generally accepted that the therapeutic potential of doxorubicin could be significantly improved by targeted drug delivery since its dangerous side effects thereby could be avoided or at least substantially reduced. The most dangerous side effect of doxorubicin is damage to the heart. When the cumulative dose of doxorubicin reaches 550 mg/m², the risk of developing cardiac side effects increases dramatically. Doxorubicin cardiotoxicity is characterized by a dose-dependent decline in mitochondrial oxidative phosphorylation. Reactive oxygen species (ROS) generated by the interaction of doxorubicin with iron can damage myocytes causing myofibrillar loss and cytoplasmic vacuolization. Excessive damage of this kind may result in the death of the patient. It is therefore desirable to keep the cardiac concentration of doxorubicin as low as possible.

Doxorubicin is widely used for the treatment of different types of malignancies in lung, e.g., small-cell lung cancer and pulmonary metastases of sarcomas, and development of targeted delivery of doxorubicin to the lung has a great therapeutic potential.

Isolated lung perfusion, the most developed approach for such targeted delivery of drugs to the lung, is a surgical procedure during which the circulation of blood to the lungs is separated from the circulation of blood through the rest of the body, and the drug is delivered directly into the lung circulation. This allows a higher concentration of chemotherapy to reach tumors in the lungs. This very invasive procedure is technically complicated and not safe for the patient. Among the complications of invasive procedures can be mentioned anesthesia or medication reactions, bleeding, infection, internal organ injury, and blood vessel injury. Thus it would be desirable to create a drug delivery system using convenient and minimally invasive intravenous mode of drug administration which nevertheless could provide an increased concentration of the drug in an organ or/and a tissue in question.

In addition to exclusive administration of a drug to a specific body site it is sometimes desirable to target it to more than one site. For instance, in solid cancer tumour treatment, it may be beneficial to keep its concentration at a high level in the tumour while maintaining a low concentration of the drug in the systemic circulation to prevent dissemination of metastases.

The same consideration is applicable for treatment of other diseases of lungs such as pneumonia, candidiasis, tuberculosis, as well as chronic obstructive pulmonary disease (COPD), also known as chronic obstructive lung disease (COLD), asthma, cystic fibrosis and other problems with health when a quick and targeted delivery of an active pharmaceutical ingredient to lung is desirable.

Many pharmacologically active agents such as the aforementioned drugs are weak bases in that they comprise one or more amino groups. For this reason they form salts with strong and weak acids, and are usually administered in salt form. The solubility of their common pharmaceutically acceptable salts, in particular their hydrochlorides, hydrobromides, phosphates, sulfates, lactates, tartrates, etc. in aqueous body fluid is usually higher than the solubility of the free base. Therefore aqueous solutions of such salts are used for intravenous infusion rather than an aqueous solution of the respective base.

For administration to a person or animal drugs of this kind are provided in a cationic amphiphilic form (in the form of a salt with a pharmaceutically acceptable acid). This manner of administration is applied but not limited to antineoplastic drugs such as e.g., anthracyclines, vinca alkaloids, amsacrine, topotecan and irinotecan.

Cationic amphiphilic drugs (CAD) of the aforementioned kind react with amphiphilic anionic surfactants, such as alkyl sulfates or alkane sulfonates, to form water insoluble complexes.

CADⁿ⁺Cl⁻ₙ+nNa⁺(RSO₃)⁻→CADⁿ⁺(RSO₃)⁻ₙ↓+nNa⁺Cl⁻

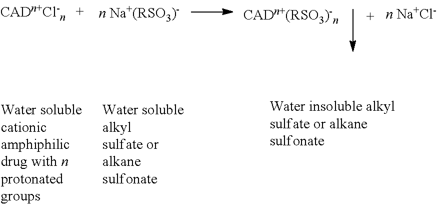

While still meeting the definition of a salt of an organic base with an organic or inorganic acid, the water insoluble complexes are to some extent additionally linked by non-covalent forces.

By using the above described concept of “programmed drug delivery” of a desired amount of a drug to be delivered to a particular tissue or organ can be designed and programmed, e.g., by a content or composition of a drug delivery formulation.

However, such techniques known and used today are in need of further improvements, in particular regarding drugs aimed to treat lung diseases, such as e.g., cancer, bacterial and fungal infections.

### OBJECTS OF THE INVENTION

A primary object of the invention is to provide a pharmaceutical composition for targeted administration to the lung of drug comprising one or more amino functions, which is lacking one or more of the drawbacks of known compositions of the drug or at least exhibits them to a lesser extent.

Another object of the invention is to provide a pharmaceutical composition for targeted administration to the lung of a drug comprising one or more amino functions, which is lacking one or more of the drawbacks of known compositions of the drug known in the art or at least exhibits them to a lesser extent.

A further object of the invention is to provide a method of designing pharmaceutical compositions of this kind that will provide, after minimally invasive intravenous mode of drug administration, a desired target concentration of the drug in the lung.

Additional objects of the invention will become evident from the study of the following short description of the invention, of preferred embodiments thereof, and of the appended claims.

### SUMMARY OF THE INVENTION

The present invention is based on the insight that aqueous suspensions of particles of amphiphilic straight chain alkyl sulfonate and of straight chain alkane sulfates of hydrophilic anti-cancer drugs comprising amino function(s) are valuable forms by which these drugs can be administered in a manner concentrating their therapeutic effect to the desired organ, in particular the lung after minimally invasive intravenous mode of drug administration. An important feature of the straight chain alkyl sulfonate and straight chain alkane sulfates of hydrophilic anti-cancer drugs is their low solubility in water and aqueous body fluid of less than 0.1 mg/mL at 25° C.

A possible explanation of the biology behind the invention, which is however in no way binding, is that upon administration of a particulate aqueous suspension of an anti-cancer drug of this kind to the systemic circulation or the lung or a solid tumour the drug particles will reach, within a given period of time, an equilibrium distribution in the body. Their solubility in aqueous media is very low but not nil. They will therefore slowly dissolve in body fluid until an equilibrium determined by their solubility is reached. Since the dissolved material is irreversibly transformed chemically to degradation products more material is dissolved over time to maintain the equilibrium. As long as the equilibrium is fed by dissolving material a steady state concentration of the drug is maintained locally.

The present invention is furthermore based on the insight that aqueous suspensions of amphiphilic straight chain alkyl sulfonates and straight chain alkane sulfates of hydrophilic anti-cancer drugs comprising amino function(s) are particularly valuable forms by which these drugs can be targeted to the lung (or accumulated in lung tissue) after minimally invasive intravenous administration. Aqueous suspensions are constituted by particles or comprise particles of a size of above about 5000 nm.

An important property of aqueous suspensions of the invention is their low sedimentation rate. In general the sedimentation rate of a given sort of particle increases with particle size. It may however be prevented from increasing and even be decreased by increasing the viscosity of the aqueous phase and/or by changing a surface property of the particles, such as, for instance, surface roughness.

The present invention provides solid particles of a salt of a hydrophilic drug comprising one or more amino groups and a water soluble alkyl sulfate or alkane sulfonate or a mixture of two or more of such sulfates or sulfonates. An important feature of the salt is its low solubility in water. In other words, the salts of the invention are substantially insoluble. By “substantially insoluble” it is understood a solubility in water or aqueous body fluid of less than 0.1% by weight, in particular of less than 0.05% by weight or 0.02% by weight.

The present invention provides a method of producing said solid particles of a water insoluble salt of a hydrophilic cancer drug with a water soluble alkyl sulfate or alkane sulfonate or with a mixture of two or more of such sulfates or sulfonates.

The present invention furthermore provides a pharmaceutical composition comprising one or more amphiphilic sulfonates and/or sulfates of the invention and a liquid carrier. The composition can be administered by any suitable route, such as by intraarterial, intraperitoneal, intramuscular, transdermal or intravenous administration. Administration by using minimally-invasive intravenous administration comprising an aqueous suspension of the amphiphilic sulfonates and sulfates of the invention is preferred.

The present invention also provides a method of producing a pharmaceutical composition comprising a water insoluble salt of a hydrophilic drug and a water soluble alkyl sulfate or alkane sulfonate or of a mixture of two or more of such sulfates or sulfonates in form of solid particles.

The composition of the invention may further comprise a buffer and pharmaceutically acceptable excipients such as osmolality controlling agent and viscosity controlling agent. Due to the method of production used the composition additionally contains a salt or corresponding ions consisting of the cation of the water soluble alkyl sulfate or alkane sulfonate and of the anion of the anti-cancer drug. The use of alkali alkyl sulfates and of alkali alkane sulfonates, in particular of sodium and potassium alkyl sulfates and alkane sulfonates, is preferred.

The amphiphilic particulate sulfonates and sulfates of the invention consist of a pharmacological agent D possessing anti-cancer activity comprising from 1 to 4 amino groups of which one or more is protonated, and of one or more sulphate or sulfonate anion. They are represented by formulas (1) and (2):

Dⁿ⁺(R¹SO₃)⁻ₙ (1)

Dⁿ⁺(R²OSO₃)⁻ₙ (2)

wherein R¹ is straight chain C₆-C₃₀ alkyl; R² is straight chain C₆-C₃₀ alkyl; n is an integer from 1 to 4.

It is preferred for R¹ and R² to be straight chain C₁₀-C₂₀ alkyl, more preferred to be straight chain C₁₂-C₁₈ alkyl, even more preferred to be about straight chain C₁₆ alkyl. In consequence, R¹ can be any of straight chain C₁₂, C₁₃, C₁₄, C₁₅, C₁₆, C₁₇, C₁₈ alkyl; R² is any of straight chain C₁₂, C₁₃, C₁₄, C₁₅, C₁₆, C₁₇, C₁₈ alkyl.

A preferred particle size of 90% of the colloid particles is within 5000 nm to 100000 nm, preferred within 20000 nm to 90000 nm, most preferred within 40000 nm to 80000 nm.

According to a preferred aspect of the invention particles of larger size than colloid particles and their aqeuous suspensions are comprised by the present invention, such as particles of a size of up to 50 μm or 100 μm, and their suspensions.

The particles of the invention can be separated from the aqueous phase by, for instance, centrifugation or cryoprecipitation. If separated by centrifugation accompanying salt or corresponding ions consisting of the cation of the water soluble alkyl sulfate or alkane sulfonate and of the anion of the anti-cancer drug are eliminated with the aqueous phase. The resulting powder (additionally dried, if necessary) retains the particle size of the colloid to at least 50%, more preferred to at least 80%. To facilitate re-suspension in an aqueous media, the powder can comprise a re-suspension facilitating agent such as glucose, lactose or albumin. Alternatively the particles of the invention can be produced by evaporation, including cryoprecipitation, of the aqueous media; in such case they will be admixed with accompanying salt comprising the cation of the water soluble alkyl sulfate or alkane sulfonate and the anion of the anti-cancer drug; if desired they can be admixed with resuspension facilitating agent.

According to another preferred aspect of the invention, suspensions of the invention can be comprised by micro carrier particles having affinity, such as by including appropriate antibody structures, to a surface antigen of the tumour to be treated.

Preferred pharmacologically active agents D of the amphiphilic sulfonates and sulfates of the invention include but are not limited to doxorubicin, epirubicin, daunorubicin, idarubicin, mitoxantrone, viniblastine, vincristine, vinorelbine, amsacrine, topotecan, irinotecan.

According to a further preferred aspect of the invention, suitable antibiotics are of similar hydrophilicity as aminoglycosides, ansamycins, carbapenems, cephalosporins, glycopeptides, daptomycin, macrolides, oxazolidinones, penicillins, quinolones (ciprofloxacin, enoxacin, gatifloxacin, gemifloxacin, levofloxacin, lomefloxacin, moxifloxacin norfloxacin, ofloxacin, trovafloxacin, grepafloxacin sparfloxacin, temafloxacin), sulfonamides, tetracyclines (doxycycline, tetracycline, minocycline, oxytetracycline), drugs against mycobacteria (clofazimine, dapsone, capreomycin, cycloserine, ethambutol, ethionamide, isoniazid, pyrazinamide, rifampicin, rifapentine, streptomycin).

Suitable antifungal drugs are of similar hydrophilicity of polyene antifungals (amphotericin B, candicidin, filipin, hamycin, natamycin, nystatin), echinocandins (anidulafungin, caspofungin, micafungin), azole antifungal drugs (i.e. imidazole, triazole, and thiazole antifungals of different structures).

According to the present invention also disclosed is a method of treating a disease in a person, comprising administrating to said person a therapeutically effective amount of the pharmaceutical composition of the invention or of a pharmaceutical composition comprising amphiphilic particulate sulfonate or sulfate powder of the invention. A preferred method of administration is by infusion or injection into a vein or artery. Another preferred method of administration is to a solid tumour or another target tissue by infusion or injection into the peripheral circulation. A third preferred method of administration is by infusion or injection directly into a solid tumour or target tissue. According to a preferred aspect of the invention administration is by a bolus or by several boli is preferred.

According to the present invention is provided a drug delivery system for convenient and minimally-invasive intravenous administration capable of providing a desired concentration of the drug in a lung over extended periods of time, such as for more than one hour or six hours or even a day or more. According to a preferred aspect of the invention the invention provides a method of controlling the ratio of distribution of a drug between a particular target organ or tissue and other organs and tissues.

Doxycycline is a broad-spectrum antibiotic of the tetracycline class that is useful for the treatment of a number of infections, including bacterial, protozoal and helminth.

As with all tetracycline antibiotics, it is contraindicated in pregnancy through infancy and childhood up to eight years of age, due to the potential for disrupting bone and tooth development. A targeted delivery of doxycycline to lung reduces the systemic toxicity and significantly improves the therapeutic profile of this drug as well as makes it available for pediatric use.

Another example is an antifungal drug Amphotericin B which is often used intravenously for pulmonary fungal infections. It is the only effective treatment for some fungal infections and it is well known for its severe and potentially lethal side effects. Very often, a serious acute reaction after the infusion is noted, consisting of high fever, shaking chills, hypotension, anorexia, nausea, vomiting, headache, dyspnea and tachypnea, drowsiness, and generalized weakness. Targeted delivery of Amphotericin B to the lung enables decrease of the dose of the drug and will therefore significantly improve efficiency and safety of a pulmonary candidiasis treatment.

According to the invention also disclosed is a method of designing a pharmaceutical composition for providing, during a predetermined period, a therapeutic target of a person or animal selected from lung, other organ, and solid tumour, with a predetermined concentration of a sulfate or sulfonate of a pharmacologically active agent D comprising from 1 to 4 amino groups represented by formula (1) or (2) or a mixture of these agents:

Dⁿ⁺(R¹SO₃)⁻ₙ (1)

Dⁿ⁺(R²OSO₃)⁻ₙ (2)

wherein R¹ is straight chain C₆-C₃₀ alkyl; R² is straight chain C₆-C₃₀ alkyl; n is an integer from 1 to 4; wherein the method comprises:
i) determining the solubility of Dₙ⁺(R¹SO₃⁻)ₙ and/or Dⁿ⁺(R²OSO₃⁻)n for various carbon chain lengths X, Y in an aqueous solvent;
ii) determining the correlation between the solubility of said sulfate or sulfonate of said pharmacologically active agent and the expected concentration of said pharmaceutically active agent D in the therapeutic target upon administration of said pharmacologically active agent D to the subject or animal;
iii) defining a target solubility of said sulfate or sulfonate of said pharmacologically active agent in said solvent based on a desired concentration of said pharmaceutically active substance D in said organ or tissue;
iv) determining the carbon chain length(s) X, Y corresponding to said target solubility;
v) providing a sulfate or sulfonate of said pharmacologically active agent comprising the so determined the carbon chain length(s) X, Y;
vi) providing a fluid carrier;
vii) combining said sulfate or sulfonate of said pharmacologically active agent comprising the so determined the carbon chain length(s) X, Y with the fluid carrier in an amount capable of maintaining said concentration during said period.

According to a preferred aspect of the invention the solubility is determined in an aqueous organic solvent, such as an aqueous alcohol, in particular aqueous ethanol in a concentration of from 5% to 50%, preferably from 10% to 30% (v/v). Other water miscible solvents and surfactants such as low molecular weight ketones, amides, esters, amides, sulfoxides and albumins may also be used.

According to a preferred aspect of the invention the pharmaceutical composition comprises a mixture of at least two different sulfates or sulfonates of the invention represented by formula (1) or (2) or at least two different sulfates or sulfonates of which one is represented by formula (1) and the other by formula (2).

A preferred pharmacologically active substance D is selected from the group consisting of but not limited to anti-cancer drugs such as, for instance for antineoplastic drugs, anthracyclines (doxorubicin, epirubicin, daunoruicin, idarubicin, mitoxantrone), vinca alkaloids (vinblastine, vincristine, vinorelbine), amsacrine, topotecan and irinotecan; for instance for antibiotics, aminoglycosides, ansamycins, carbapenems, cephalosporins, glycopeptides, daptomycin, macrolides, oxazolidinones, penicillins, quinolones (ciprofloxacin, enoxacin, gatifloxacin, gemifloxacin, levofloxacin, lomefloxacin, moxifloxacin norfloxacin, ofloxacin, trovafloxacin, grepafloxacin sparfloxacin, temafloxacin), sulfonamides, tetracyclines (doxycycline, tetracycline, minocycline, oxytetracycline), drugs against mycobacteria (clofazimine, dapsone, capreomycin, cycloserine, ethambutol, ethionamide, isoniazid, pyrazinamide, rifampicin, rifapentine, streptomycin); for instance for antifungal drugs, polyene antifungals (amphotericin B, candicidin, filipin, hamycin, natamycin, nystatin), echinocandins (anidulafungin, caspofungin, micafungin), azole antifungal drugs (i.e. imidazole, triazole, and thiazole antifungals of different structures). It is also preferred for the composition to comprise a suspension.

A preferred fluid carrier is water or an aqueous media in which the sulfate or sulfonate of said pharmacologically active agent D is insoluble or substantially insoluble. By “substantially insoluble” is understood a solubility of less than 0.1% by weight, in particular of less than 0.05 or 0.02 by weight. The composition may be designed for intraarterial, intraperitoneal, intramuscular, transdermal or intravenous administration. The steps above may be performed in any suitable order.

A preferred form of said sulfate or sulfonate of the pharmacologically active agent D is a powder or a suspension of a mean particle size (N) in interval 5 μm to 100 μm. More preferred between 20 μm and 90 μm, or 40 μm and 80 μm. A preferred form of said pharmaceutical composition is an aqueous suspension.

According to the present invention is also disclosed a method of producing the pharmaceutical composition of the invention, the method comprising: providing a first aqeuous solution of a salt of said drug (D) with an inorganic or organic acid that is not amphiphilic; providing a second aqueous solution comprising an amount of a sodium or potassium salt of an alkyl sulfonate of the formula (Na or K)⁺(R¹SO₃)⁻ or of an alkane sulfate of the formula (Na or K)⁺(R₂OSO₃)⁻ equivalent to the amount of said salt; mixing said first and second solutions. While other than sodium and potassium salts can be used in the method, their use is not preferred. It is preferred for R¹ to be straight chain C₆-C₃₀ alkyl; R² is straight chain C₆-C₃₀ alkyl; n is an integer from 1 to 4. It is preferred for R¹ and R² to be straight chain C₁₀-C₂₀ alkyl, more preferred straight chain C₁₂-C₁₈ alkyl, most preferred about straight chain C₁₂-C₁₆ alkyl.

Further the present invention provides said pharmacological composition or amphiphilic particulate sulfonate or sulfate powder for use in treating a lung disease. The diseases may be cancer, fungal or bacterial infections/diseases.

The invention will now be illustrated in greater detail by a number of non-limiting examples thereof.

### BRIEF DESCRIPTION OF THE DRAWINGS

FIG. 1 is a graph illustrating the dependence of the solubility of doxorubicin alkyl sulfate in 30% aqueous ethanol on alkyl chain length;

FIG. 2 is a graph illustrating the dependence of the solubility of doxorubicin alkane sulfonate in 30% aqueous ethanol on alkyl chain length;

FIG. 3 is a graph illustrating the dependence of the solubility of mitoxantrone alkyl sulfate in 30% aqueous ethanol on alkyl chain length;

FIG. 4 is a graph illustrating the dependence of the solubility of mitoxantrone alkane sulfonate in 30% aqueous ethanol on alkyl chain length;

FIG. 5 is a graph illustrating the dependence of the solubility of irinotecan alkane sulfonate in 10% aqueous ethanol on alkyl chain length;

FIG. 6 is a graph illustrating the dependence of the solubility of vinorelbine alkane sulfonate in 20% aqueous ethanol on alkyl chain length;

FIG. 7 is a graph illustrating the dependence of the solubility of doxycycline complex with alkane sulfonates in 20% aqueous ethanol on the length of alkyl chain of the alkane sulfonates.

FIG. 8 is a graph illustrating the dependence of the solubility of doxycycline complex with alkyl sulfates in 20% aqueous ethanol on the length of alkyl chain of the alkyl sulfates

FIG. 9 is a graph illustrating the dependence of the solubility of amphotericin B complex with alkane sulfonates in 30% aqueous ethanol on the length of alkyl chain of the alkane sulfonates

FIG. 10 is a graph illustrating the dependence of the concentration of doxorubicin in lung in Wistar rats on the solubility of complexes in 30% aqueous ethanol

FIG. 11 is a graph illustrating the dependence of the concentration of doxorubicin in lung in Californian rabbits on the solubility of complexes in 30% aqueous ethanol.

FIG. 12 is a graph illustrating the dependence of the concentration of doxycycline in lung in Wistar rats on the solubility of complexes in 20% aqueous ethanol.

FIG. 13 is a graph illustrating the dependence of the concentration of doxycycline in blood serum in Wistar rats on the solubility of complexes in 20% aqueous ethanol.

FIG. 14 is a graph showing the relationship between amount drug, i.e., doxorubicin measured in lung and average particle size of the complex.

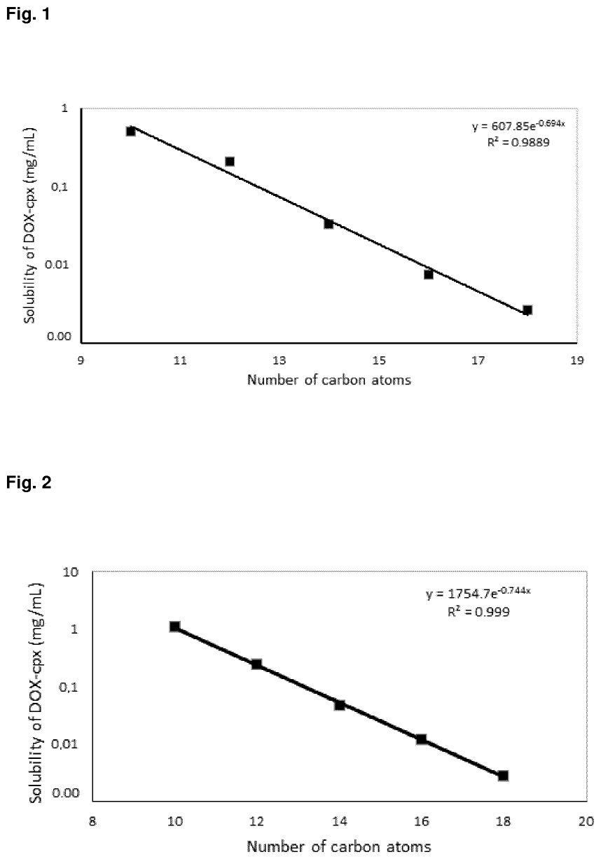

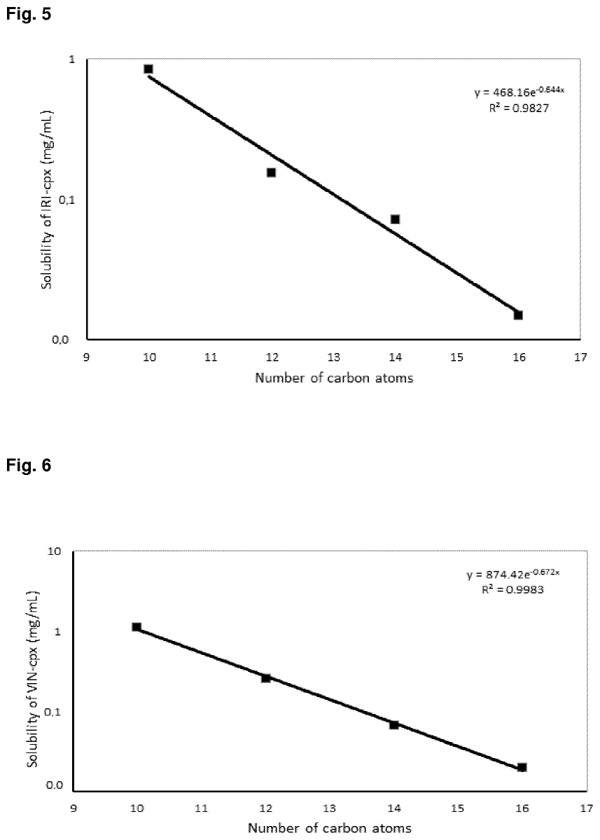

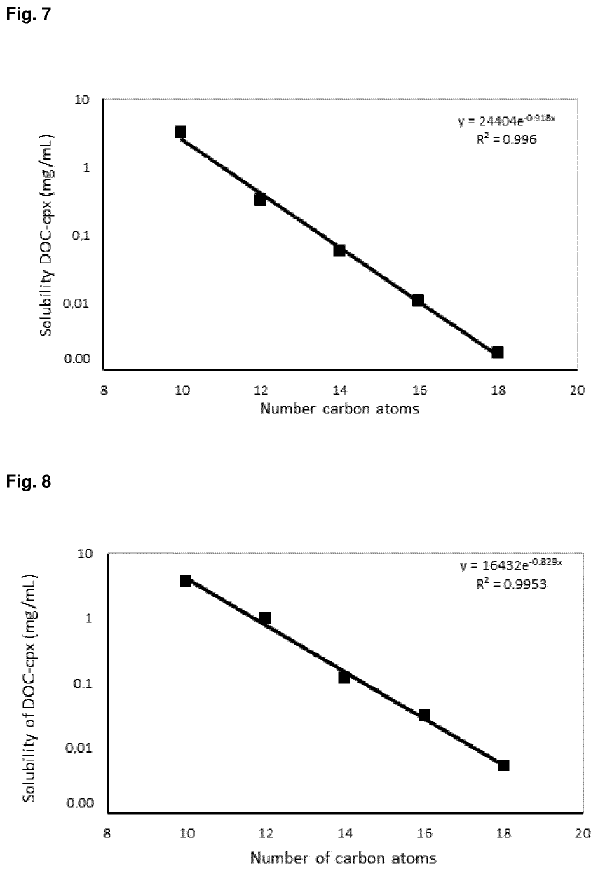

Exponential (for FIG. 1-9) and logarithmic (for FIG. 10-13) trendlines and their equations were obtained with the use of Microsoft Excel software.

### DETAILED DESCRIPTION

It is to be understood that this invention is not limited to the particular configurations, process steps, and materials disclosed herein as such configurations, process steps, and materials may vary somewhat. It is also to be understood that the terminology employed herein is used for the purpose of describing particular embodiments only and is not intended to be limiting since the scope of the present invention will be limited only by the appended claims and equivalents thereof.

All references cited herein are incorporated herein by reference in their entirety and for all purposes to the same extent as if each individual publication or patent or patent application was specifically and individually indicated to be incorporated by reference in its entirety for all purposes.

The present invention is best understood by reference to the following definitions, the Figures and exemplary disclosure provided herein.

In this application, unless otherwise stated, the term “lung disease” comprises primary Acute Bronchitis, Acute Respiratory Distress Syndrome (ARDS), Asbestosis, Asthma, Bronchiectasis, Bronchiolitis, Bronchiolitis Obliterans Organizing Pneumonia (BOOP), Bronchopulmonary Dysplasia, Byssinosis, Chronic Bronchitis, Coccidioidomycosis (Cocci), Chronic Obstructive Pulmonary Disease (COPD), Cryptogenic Organizing Pneumonia (COP), Cystic Fibrosis, Emphysema, Hantavirus Pulmonary Syndrome, Histoplasmosis, Human Metapneumovirus, Hypersensitivity Pneumonitis, Influenza, Middle Eastern Respiratory Syndrome, Nontuberculosis Mycobacterium, Pertussis, Pneumoconiosis (Black Lung Disease), Pneumonia of different origin, Primary Ciliary Dyskinesia, Primary Pulmonary Hypertension, Pulmonary Arterial Hypertension, Pulmonary Fibrosis, Pulmonary Vascular Disease, Respiratory Syncytial Virus, Sarcoidosis, Severe Acute Respiratory Syndrome, Silicosis, Sleep Apnea, and Sudden Infant Death Syndrome.

For example doxycycline (DOX) is a broad-spectrum antibiotic of the tetracycline class that is useful for the treatment of a number of infections, including bacterial, protozoal and helminth.

Another example is an antifungal drug Amphotericin B (ampB) which is often used intravenously for pulmonary fungal infections. It is the only effective treatment for some fungal infections and it is well known for its severe and potentially lethal side effects.

In this application, unless otherwise stated, the term lung cancer comprises primary cancer such as non-small cell lung cancer, small cell lung cancer as well as secondary tumors in a lung, Lymphangiomatosis, Mesothelioma.

Materials and Methods

Solubility in aqueous ethanol was determined by centrifuging an adequate amount of freshly obtained colloid at 3000 rpm for 30-90 min, decanting the supernatant, adding 10 mL water and shaking the mixture, then repeating centrifugation, shaking and washing 3 times. The centrifugate from the final centrifugation was air dried for 72 h at room temperature followed by drying in vacuo for 24 h. A portion of the dried centrifugate (20 mg) was resuspended in 6 mL aqueous ethanol (EtOH) by stirring at room temperature for 24 h. The mixture was centrifuged at 3000 rpm for 10 min and the supernatant filtered through a 0.2 micrometer filter to remove aggregates of undissolved solid product. The solubility of the compound was determined by a UV method. Particle size analysis was accomplished by laser diffraction method.

The composition used for in vivo investigation was freshly prepared or obtained by dilution of concentrates. For in vivo investigation both rats and rabbits were used: female Wistar rats 60-75 days old weighing 300 g±30 g and Californian breed male rabbits 75-90 days old weighing 2000 g±250 g were selected. For every formulation tested at a particular time point, 4 rats or 3 rabbits were used. Different doxorubicin containing formulations with a total doxorubicin dose 5 mg/kg were administered via a single bolus injection into the tail for rats and via a slow flow of 1 ml/min in the marginal ear vein for rabbits. Immediately after sacrification, the animal organs and tissues were deep-frozen in liquid nitrogen.

Determination of the Bio-Distribution of Doxorubicin and Doxycycline in Lung Tissue.

Five or six pieces of lung tissue of a total weight of about 1 g were taken from different parts of a lung. The samples were homogenized with a solution of aqueous ethanol containing HCl for 20 s at 7000 rpm and for 10 s at 11000 rpm. The homogenate obtained was vortexed for 30 min and centrifuged at 3000 rpm for 30 min. The supernatant was treated with a solution of monochloroacetic acid and incubated for 1 hour followed by centrifugation of the mixture obtained at 15000 rpm for 15 min. Doxorubicin and doxycycline (DOC) concentration in the final supernatant was determined with fluorometric analysis and high-performance liquid chromatography respectively.

### Example 1

Preparation of Suspension of Doxorubicin Alkyl Sulfate and Alkane Sulfonate

To a solution of doxorubicin hydrochloride (DOX Cl) (50 mL, 1 mg/mL) in 5% aqueous dextrose in an Erlenmeyer flask was added at room temperature a solution of a 5-10% molar excess of Na⁺(R¹SO₃)⁻ or Na⁻(R²OSO₃)⁻ and in the same solvent as for doxorubicin hydrochloride. Instead of 5% aqueous dextrose can be used in this and the other examples Ringer solution or 0.9% saline or phosphate-buffered saline or another aqueous solution of an osmolality from 270 to 300 mOsm/L. The process of colloid formation was monitored visually. After completing of the addition the mixture was vortexed or shaken for an additional time period varying from 30 min to 7 days. The suspension obtained then was either directly used or placed for storage in a refrigerator. Concentration of doxorubicin in the compositions was determined by a UV method at 495 or 233 nm. For sampling, an aliquot of the colloid was diluted with methanol (excess of methanol>20:1).

### Example 2

Preparation of Suspension of Mitoxantrone (MIT) Alkyl Sulfates and Alkane Sulfonates

To a vigorously stirred solution of mitoxantrone dihydrochloride (40 mL, 0.2 mg/mL) in 5% dextrose in water in an Erlenmeyer flask was added at room temperature a solution containing 0.03 mmol of Na⁺(R¹SO₃)⁻ or Na⁺(R²OSO₃)⁻ in the same solvent as for mitoxantrone dihydrochloride. The formation of a black colloid was monitored visually. The colloid slowly disintegrated into a black precipitate and a pale supernatant. After completing of the addition the mixture obtained stirred for additional time (from 1 to 7 days). The suspension composition was either used directly or stored in a refrigerator for later use. The concentration of mitoxantrone in the colloid was determined by a UV method at 662, 611 or 242 nm. For sampling an aliquot of the colloid was diluted with methanol to >20:1.

### Example 3

Preparation of Suspension of Irinotecan (IRI) Alkyl Sulfates and Alkane Sulfonates

To a vigorously solution of irinotecan hydrochloride trihydrate (5 mL, 4 mg/mL) in deionized water was added at room temperature a solution containing Na⁺(R¹SO₃)⁻ or Na⁺(R²OSO₃)⁻ in deionized water. The formation of a colloid was monitored visually. After completing of the addition the mixture obtained stirred for 2 days and the mixture was centrifuged 10 min at 3000 rpm. On standing the white colloid slowly disintegrated into a white precipitate and a nearly colourless supernatant. The supernatant was replaced by 5% aqeuous dextrose. The precipitate was resuspended in water by vortexing for 10 min. The composition obtained then was either directly used or stored in a refrigerator for future use. The concentration of irinotecane in the colloid or suspension was determined by a UV method at 360, 255 or 220 nm. For sampling, an aliquot of the product was diluted with methanol (excess of methanol>20:1).

### Example 4

Preparation of Suspension of Vinorelbine (VIN) Alkyl Sulfates and Alkane Sulfonates

To a vigorously stirred solution of vinorelbine tartrate (2 mL, 5 mg/mL) in 5% aqueous dextrose in an Erlenmeyer flask was added at room temperature a solution of one equivalent of Na⁺(R¹SO₃)⁻ or Na⁺(R²OSO₃)⁻ in the same solvent as for vinorelbine tartrate. The formation of a colloid was monitored visually. After completing of the addition the mixture obtained was vortexed or shaken for 7 days. On standing the colloid slowly disintegrated into a precipitate and a clear supernatant. The suspension was either used directly or stored in a refrigerator for future use. The concentration of vinorelbine in the suspension was determined by a UV method at 268 or 212 nm. For sampling, an aliquot of the colloid was diluted with methanol (excess of methanol>20:1).

### Example 5

Preparation of Suspension of Doxycycline (DOC) Alkyl Sulfates and Alkane Sulfonates

To a solution of doxycycline hyclate (50 mL, 1 mg/mL) in 5% aqueous dextrose in an Erlenmeyer flask was added at room temperature a solution of a 5-10% molar excess of Na⁺(R¹SO₃)⁻ or Na⁺(R²OSO₃)⁻ and in the same solvent as for doxycycline hyclate hydrochloride. Instead of 5% aqueous dextrose can be used in this and the other examples Ringer solution or 0.9% saline or phosphate-buffered saline or another aqueous solution of an osmolality from 270 to 300 mOsm/L. The process of colloid formation was monitored visually. After completing of the addition the mixture was vortexed or shaken for an additional time period varying from 30 min to 7 days. The suspension obtained then was either directly used or placed for storage in a refrigerator. Concentration of doxycycline in the compositions was determined by a UV method at 273 and 345 nm. For sampling, an aliquot of the colloid was diluted with methanol (excess of methanol>20:1).

### Example 6

Preparation of Suspension of Amphotericin B (ampB) Alkyl Sulfates and Alkane Sulfonates

To a solution of amphotericin B (2 mL, conc 0.5 mg/mL) in 5% aqueous dextrose in an Erlenmeyer flask was added at room temperature a solution of a 5-10% molar excess of Na⁺(R¹SO₃)⁻ or Na⁺(R²OSO₃)⁻ and in the same solvent as for amphotericin. Instead of 5% aqueous dextrose can be used in this and the other examples Ringer solution or 0.9% saline or phosphate-buffered saline or another aqueous solution of an osmolality from 270 to 300 mOsm/L. The process of colloid formation was monitored visually. After completing of the addition the mixture was vortexed or shaken for an additional time period varying from 30 min to 3 days. The suspension obtained then was either directly used or placed for storage in a refrigerator. Concentration of amphotericin in the compositions was determined by a UV method at 410 or 385 nm. For sampling, an aliquot of the colloid was diluted with methanol (excess of methanol>20:1).

### Example 7

Solubility of Suspension of Doxorubicin (DOX) Alkyl Sulfates and Alkane Sulfonates in 30% Aqueous Ethanol

Solubility was determined in accordance with the general method described under Materials and Methods. The results are summarized in Table 1 and presented in FIGS. 1 and 2.

TABLE 1
Solubility of suspension of doxorubicin alkyl
sulfates and alkane sufonates in 30% ethanol (v/v).

| Generic formula | Alkyl chain | Solubility, |
|---|---|---|
| of anion | length, n | mg/mL |
| CₙH₂ₙ₊₁OSO₃⁻ | 10 | 0.50769 |
| | 12 | 0.20560 |
| | 14 | 0.03281 |
| | 16 | 0.00729 |
| | 18 | 0.00260 |
| CₙH₂ₙ₊₁SO₃⁻ | 10 | 1.06429 |
| | 12 | 0.23953 |
| | 14 | 0.04596 |
| | 16 | 0.01194 |
| | 18 | 0.00279 |

### Example 8

Solubility of Suspension of Mitoxantrone Alkyl Sulfates and Alkane Sulfonates in 30% Aqueous Ethanol

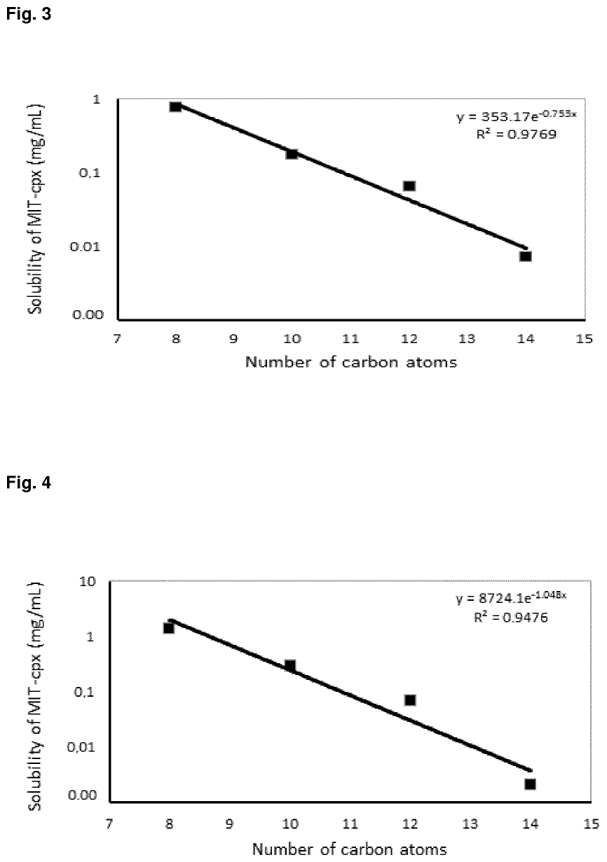

The solubility was determined in accordance with the general method described under Materials and Methods. The results are summarized in Table 2 and presented in FIGS. 3 and 4.

TABLE 2
Solubility of suspension of mitoxantrone alkyl
sulfates and alkane sufonates in 30% ethanol (v/v).

| Generic formula | Alkyl chain | Solubility |
|---|---|---|
| of anion | length (n) | (mg/mL) |
| CₙH₂ₙ₊₁OSO₃⁻ | 8 | 0.78058 |
| | 10 | 0.17582 |
| | 12 | 0.06484 |
| | 14 | 0.00720 |
| CₙH₂ₙ₊₁SO₃⁻ | 8 | 1.34798 |
| | 10 | 0.29487 |
| | 12 | 0.68313 |
| | 14 | 0.00203 |

### Example 9

Solubility of Irinotecan (IRI) Alkane Sulfonates in 10% Aqueous Ethanol

The solubility was determined in accordance with the general method described under Materials and Methods. The results are summarized in Table 3.

TABLE 3
Solubility of suspension of irinotecan
alkane sufonates in 30% ethanol (v/v).

| Alkyl chain | Solubility, |
|---|---|
| length, n | mg/mL |
| 10 | 0.83483 |
| 12 | 0.15453 |
| 14 | 0.07064 |
| 16 | 0.01476 |

### Example 10

Solubility of Suspension of Vinorelbine Alkane Sulfonates (VIN) in 20% Aqueous Ethanol.

The solubility was determined in accordance with the general method described under Materials and Methods. The results are summarized in Table 4 and visualized in FIG. 6.

TABLE 4
Solubility of suspension of vinorelbine alkane
sulfonates in 20% aqueous ethanol.

| Alkyl chain | Solubility, |
|---|---|
| length, n | mg/mL |
| 10 | 1.11868 |
| 12 | 0.25692 |
| 14 | 0.06731 |
| 16 | 0.01977 |

### Example 11

Solubility of Suspension of Doxycycline (DOC) Alkane Sulfonates and Alkyl Sulfates in 20% Aqueous Ethanol

The solubility was determined in accordance with the general method described under Materials and Methods. The results are summarized in Table 5.

### Example 12

Solubility of Suspension of Amphotericin B (ampB) Alkane Sulfonates in 30% Aqueous Ethanol

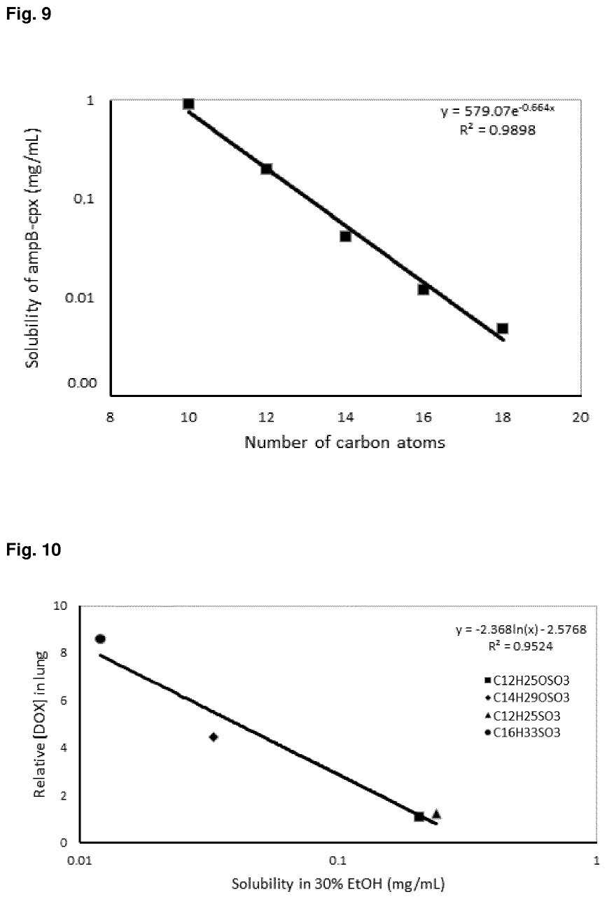

The solubility was determined in accordance with the general method described under Materials and Methods. The results are summarized in Table 6 and illustrated in FIG. 9.

TABLE 5
Solubility of suspension of doxycycline (DOC) alkane
sulfonates and alkyl sulfates in 20% aqueous ethanol.

| Generic formula | Length of C | Solubility, |
|---|---|---|
| of anion | chain, n | mg/mL |
| CₙH₂ₙ₊₁OSO₃⁻ | 10 | 3.77651 |
| | 12 | 0.98191 |
| | 14 | 0.11776 |
| | 16 | 0.03199 |
| | 18 | 0.00523 |
| CₙH₂ₙ₊₁SO₃⁻ | 10 | 3.16529 |
| | 12 | 0.32034 |
| | 14 | 0.05510 |
| | 16 | 0.01034 |
| | 18 | 0.00181 |

TABLE 6

| Generic formula | Length of | Solubility, |
|---|---|---|
| of anion | a chain, n | mg/mL |
| CₙH₂ₙ₊₁OSO₃⁻ | 10 | 0.50769 |
| | 12 | 0.20560 |
| | 14 | 0.03281 |
| | 16 | 0.00729 |
| | 18 | 0.00260 |

### Example 13 and 14

In Vivo Analysis of Distribution of Doxorubicin (DOX) in Lung in Wistar Rats

Relationship Between Solubility of of Doxorubicin Sulfonates in 30% Aqueous Ethanol and Increase of Doxorubicin Concentration in Lung in Wistar Rats after 4 Hours after Intravenous Single Bolus Injection, (See FIG. 10).

An aqueous solution of doxorubicin hydrochloride, an aqueous suspension of doxorubicin alkane sulfate and an aqueous suspension of doxorubicin alkyl sulfonate were administered via a single bolus injection into the tail Animals were sacrificed after 4 hours after administered via a single bolus injection into the tail; total doxorubicin dose 5 mg/kg. The concentration of doxorubicin in a lung was determined according to the procedure described above. The results are summarized in Table 7 and illustrated in FIG. 10. As it is seen from the table the concentration of doxorubicin determined in blood serum does not depend on the nature of doxorubicin salt.

TABLE 7
The amount of Doxorubicin (DOX) is expressed in μg/kg ± SD, and the
difference is expressed relative to the control CI-DOX (1).EXAMPLE 14

| Anion of | Lung tissue | | Blood serum | | Solubility |
|---|---|---|---|---|---|
| doxorubicin | | Relative | | Relative | in 30% |
| salt | DOX | difference | DOX | difference | ethanol |
| CI (DOX) | 0.71 ± 0.05 | (1) | 0.019 ± 0.004 | (1) | — |
| C₁₂H₂₅OSO₃ | 0.77 ± 0.19 | 1.08 | 0.021 ± 0.007 | 1.12 | 0.206 |
| C₁₄H₂₉OSO₃ | 3.17 ± 0.33 | 4.46 | 0.023 ± 0.009 | 1.21 | 0.033 |
| C₁₂H₂₅SO₃ | 0.88 ± 0.09 | 1.24 | 0.014 ± 0.003 | 0.75 | 0.240 |
| C₁₆H₃₃SO₃ | 6.11 ± 0.75 | 8.61 | 0.019 ± 0.007 | 1.04 | 0.012 |

### Example 15

In Vivo Analysis of Distribution of Doxorubicin (DOX) in Lung in Californian Rabbits

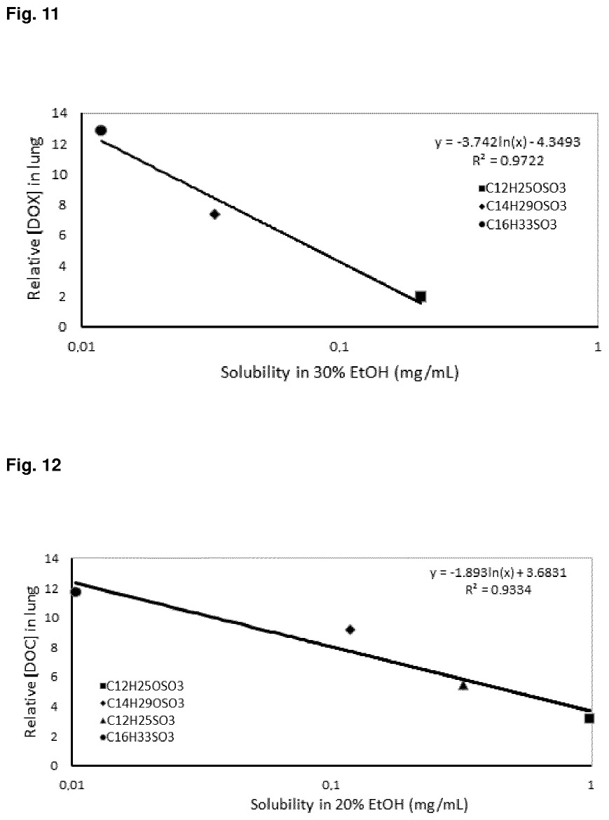

Suspension of complexes of doxorubicin and doxorubicin hydrochloride were administered via a slow flow of 1 ml/min to rabbits in the marginal ear vein with a total doxorubicin dose 1.25 mg/kg. Animals were sacrificed after 4 hours after administration. Concentration of doxorubicin in lung and femoral muscle was determined in accordance to the procedure described above. The results are summarized in Table 8 and FIG. 11. As it is seen from table 8 the concentration of doxorubicin in femoral muscle as a representation of a system concentration is not increased for complexes compared with doxorubicin hydrochloride.

TABLE 8
The amount of Doxorubicin (DOX) is expressed in μg/kg ± SD, and the
difference is expressed relative to the control CI-DOX (1).

| | Lung tissue | | Femoral muscle | | Solubility |
|---|---|---|---|---|---|
| Anion of | | Relative | | Relative | in 30% |
| DOX salt | DOX | difference | DOX | difference | ethanol |
| CI (DOX) | 0.83 ± 0.05 | (1) | 0.23 ± 0.02 | (1) | — |
| C₁₂H₂₅OSO₃ | 1.61 ± 0.25 | 1.94 | 0.20 ± 0.02 | 0.87 | 0.2056 |
| C₁₄H₂₉OSO₃ | 6.14 ± 0.53 | 7.40 | 0.06 ± 0.01 | 0.26 | 0.0328 |
| C₁₆H₃₃SO₃ | 10.70 ± 0.77 | 12.89 | 0.12 ± 0.06 | 0.52 | 0.0119 |

### Example 16

Illustrates the relationship between solubility of non-covalent complexes of doxorubicin in 30% aqueous ethanol and increase of doxorubicin concentration in lung of Californian rabbits after 4 hours after intravenous injection, See FIG. 11.

### Example 17

In Vivo Analysis of Distribution of Doxycycline (DOX) in Lung and Blood Serum in Wistar Rats

Suspension of complexes of doxycycline and doxycycline hyclate were administered via a single bolus injection into the tail with a total doxycycline dose 3 mg/kg. Animals were sacrificed after 30 min after administration. Concentration of doxycycline in a lung and blood serum was determined in accordance to the procedure described above. The results are summarized in a Table 9. As it is seen from the table the concentration of doxycycline in a blood serum is decreased for non-covalent complexes compared to doxycycline hyclate.

### Example 18

Shows an illustration of relationship between solubility of complexes of doxycycline (DOC) in 20% aqueous ethanol and increase of doxycycline concentration in lung of Wistar rats after 30 min after intravenous single bolus injection, see FIG. 12.

### Example 19

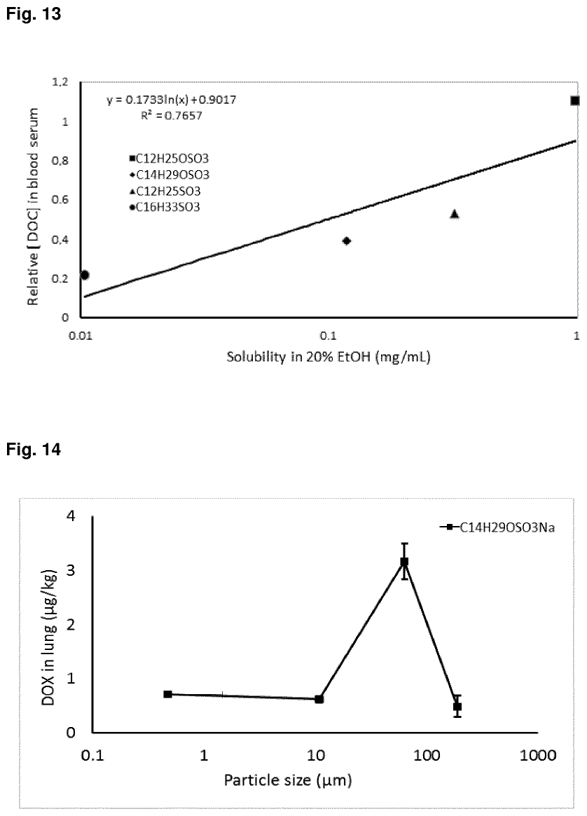

Illustrates the relationship between solubility of complexes of doxycycline in 20% aqueous ethanol and decrease of doxycycline concentration in blood serum of Wistar rats after 30 min after intravenous single bolus injection, see FIG. 13.

TABLE 9
The amount of Doxorubicin (DOX) is expressed in ng/ml ± SD, and the
difference is expressed reative to the control CI-DOX (1).

| | | | | | Solubility |
|---|---|---|---|---|---|
| Anion of | Lung tissue | | Blood serum | | in 20% |
| DOX salt | DOX | Difference | DOX | Difference | ethanol |
| CI (Dox) | 172 ± 18 | (1) | 115 ± 12 | (1) | — |
| C₁₂H₂₅OSO₃ | 551 ± 185 | 3.20 | 127 ± 22 | 1.10 | 0.9819 |
| C₁₄H₂₉OSO₃ | 1582 ± 101 | 9.20 | 45 ± 1 | 0.39 | 0.1178 |
| C₁₂H₂₅SO₃ | 943 ± 600 | 5.48 | 61 ± 7 | 0.53 | 0.3203 |
| C₁₆H₃₃SO₃ | 2020 ± 117 | 11.74 | 25 ± 2 | 0.22 | 0.0103 |

### Example 20

Preparation of a colloid non-covalent complex of doxorubicin (DOX) with desired solubility in 30% aqueous ethanol (EtoH).

This example illustrates preparation of non-covalent complexes with defined solubility in a special solvent.

The task: to prepare a colloid non-covalent complex of doxorubicin with a use of alkane sulfonates with even number of carbon atoms in 30% aqueous ethanol with solubility 0.1 mg/mL.

We assume that an impact of the number of carbon atoms in alkane sulfonate radical is additive. We suppose also a continuous function for solubility y in 30% aqueous ethanol

Following function (f1), which was obtained in example 5 (see FIG. 2) represents relationship between number of carbon atoms x and solubility y.

y=f1(x)=1754.71710 exp(−0.74429x) (eq. 1)

Using following function (f2) it is possible to perform reverse calculation, i.e. calculate number of carbon atoms X from a given solubility Y:

x=f2(y)=−1/0.7442855697 ln(y/1754.71709855) (eq 2)

For the solubility of y=0.1 mg/mL the function f2 returns x equal to 13.20628. Taking the assumption of an additive behaviour of carbon atoms in the radicals we can calculate a ratio of C12 and C14 sulfonates (the adjacent sulfonates with closest solubility) to provide the suggested C13.20628 radical:
1 equivalent of C13.20628 is equal to a mixture of 0.397 equivalents of C₁₂ and 0.603 equivalents of C₁₄.

A colloid complex with the determined ratio of C₁₂ and C₁₄ sulfonates was prepared in accordance the typical method described in example 1. The solubility of the complex was determined in accordance with the general method which is described above and was found 0.098713 mg/mL.

### Example 21

Relationship between detected amount doxorubicin (μg/kg) in lung of Wistar rats and particle size of suspensions. Aqueous suspensions of complex comprising doxorubicin and sulfate having 14 carbons i.e., C₁₄H₂₉OSO₃Na-complex with a different particle size were administered via a single bolus injection into the tail. Animals were sacrificed 4 hours after administration; total amount of doxorubicin was 5 mg/kg. The concentration of doxorubicin in lung was determined according to the procedure described above. The results are summarized in FIG. 14 and Table 10. The results show that a particle size of about 40-90 μm is particularly advantageous in targeting lung tissue.

TABLE 10

| | | | | |
|---|---|---|---|---|
| Average size of | 0.475 | 10.87 | 63.24 | 189 |
| particles (μm) | | | | |
| Doxorubicin in | 710 ± 0.0 | 620 ± 0.1 | 3170 ± 0.3 | 49 ± 0.2 |
| lung (μg/kg) | | | | |

1. A pharmaceutical composition in form of an aqueous suspension comprising solid particles of amphiphilic particulate sulfonate and/or sulfate, consisting of a pharmacologically active agent D comprising from 1 to 4 amino groups of which one or more is protonated, and of a corresponding number of sulfate or sulfonate anion of a hydrophilic drug, the particles having a solubility in water or aqueous body fluid of less than 0.1% by weight, and wherein 90% or more of the particles have a size in the interval of 5000 nm to 100 000 nm, wherein the amphiphilic particulate sulfonate or sulfate are represented by formulas (1) and (2), respectively:
Dⁿ⁺(R¹SO₃)⁻ₙ (1);
Dⁿ⁺(R₂OSO₃)⁻ₙ (2);
wherein R¹ is straight chain C₁₀-C₂₀ alkyl; R² is straight chain C₁₀-C₂₀ alkyl; n is an integer from 1 to 4, and D is selected from the group consisting of anti cancer drugs, anti bacterial drugs and anti fungal drugs.

2. The composition of claim 1, further comprising buffer and/or pharmaceutically acceptable excipient.

3. The composition of claim 1, wherein R¹ and R² is straight chain C₁₂-C₁₈ alkyl.

4. A method of producing the pharmaceutical composition of claim 1, comprising: providing a first aqeuous solution of a salt of said drug with an inorganic or organic acid that is not amphiphilic; providing a second aqueous solution comprising an amount of a sodium or potassium salt of an alkyl sulfonate of the formula (Na or K)⁺(R¹SO₃)⁻ or of an alkane sulfate of the formula (Na or K)⁺(R²OSO₃)⁻ equivalent to the amount of said salt; mixing said first and second solutions.

5. The method of claim 4, wherein R¹ is straight chain C₁₀-C₂₀ alkyl; R² is straight chain C₁₀-C₂₀ alkyl; n is an integer from 1 to 4.

6. The method of claim 5, wherein R¹ and R² is straight chain C₁₂-C₁₈ alkyl.

7. An amphiphilic particulate sulfonate or sulfate powder consisting of or comprising a pharmacologically active agent D comprising from 1 to 4 amino groups of which one or more is protonated and of a number of sulfate or sulfonate anion corresponding to the number of protonated amino groups, represented by formulas (1) and (2):
Dⁿ⁺(R¹SO₃)⁻ₙ (1)
Dⁿ⁺(R²OSO₃)⁻ₙ (2)
wherein R¹ is straight chain C₁₀-C₂₀ alkyl; R² is straight chain C₁₀-C₂₀ alkyl; n is an integer from 1 to 4.

8. The amphiphilic particulate sulfonate or sulfate powder of claim 7, wherein R¹ and R² is straight chain C₁₂-C₁₈ alkyl.

9. The amphiphilic particulate sulfonate or sulfate powder of claim 7, wherein the particle size is up to 100 μm.

10. The amphiphilic particulate sulfonate or sulfate powder of claim 7 wherein the particle size is within the range of 20 μm-90 μm, or 40 μm-80 μm.

11. The amphiphilic particulate sulfonate or sulfate powder of claim 7 comprising a re-suspension facilitating agent and/or sodium or potassium salt of hydrochloric or hydrobromic acid.

12. A method of designing a pharmaceutical composition for providing, during a predetermined period, a therapeutic target with a predetermined concentration of a sulfate or sulfonate of a pharmacologically active agent D comprising from 1 to 4 amino groups represented by formula (1) or (2) or a mixture of these agents:
Dⁿ⁺(R¹SO₃)⁻ₙ (1)
Dⁿ⁺(R²OSO₃)⁻ₙ (2)
wherein R¹ is straight chain C₁₀-C₂₀ alkyl; R² is straight chain C₁₀-C₂₀ alkyl; n is an integer from 1 to 4; wherein the method comprises:
i) determining the solubility of Dⁿ⁺(R¹SO₃)⁻ₙ and/or Dⁿ⁺(R²OSO₃)⁻ₙ for various carbon chain lengths X, Y in an aqueous solvent;
ii) determining the correlation between the solubility of said sulfate or sulfonate of said pharmacologically active agent and the expected concentration of said pharmaceutically active agent D in the therapeutic target upon administration of said pharmacologically active agent D to the subject or animal;
iii) defining a target solubility of said sulfate or sulfonate of said pharmacologically active agent in said solvent based on a desired concentration of said pharmaceutically active substance D in tissue of a lung;
iv) determining the carbon chain length(s) X, Y corresponding to said target solubility;
v) providing a sulfate or sulfonate of said pharmacologically active agent comprising the so determined carbon chain length(s) X, Y;
vi) providing a fluid carrier;
vii) combining said sulfate or sulfonate of said pharmacologically active agent comprising the so determined carbon chain length(s) X, Y with the fluid carrier in an amount capable of maintaining said concentration during said period.

13. The method of claim 12, wherein the solubility is determined in an aqueous organic solvent in particular aqueous ethanol in a concentration of from 5% to 50% (v/v).

14. The method of claim 12, wherein D is selected from the group consisting of doxorubicin, epirubicin, daunorubicin, idarubicin, mitoxantrone, viniblastine, vincristine, vinorelbine, amsacrine, topotecan, irinotecan.

15. The method of claim 12, wherein D is selected from the group consisting of aminoglycosides, ansamycins, carbapenems, cephalosporins, glycopeptides, daptomycin, macrolides, oxazolidinones, penicillins, quinolones, sulfonamides, doxycycline, tetracycline, minocycline, oxytetracycline, clofazimine, dapsone, capreomycin, cycloserine, ethambutol, ethionamide, isoniazid, pyrazinamide, rifampicin, rifapentine, streptomycin, amphotericin B, candicidin, filipin, hamycin, natamycin, nystatin, echinocandins, imidazole, triazole, and thiazole.

16. A method of treating a lung disease in a person, comprising administrating to said person a therapeutically effective amount of a pharmaceutical composition in form of an aqueous suspension comprising solid particles of amphiphilic particulate sulfonate and/or sulfate, comprising a pharmacologically active agent D comprising from 1 to 4 amino groups of which one or more is protonated, and of a corresponding number of sulfate or sulfonate anion of a hydrophilic drug, the particles having a solubility in water or aqueous body fluid of less than 0.1% by weight, and wherein 90% or more of the particles have a size in the interval of 5000 nm to 100 000 nm, wherein the amphiphilic particulate sulfonate or sulfate are represented by formulas (1) and (2), respectively:
Dⁿ⁺(R¹SO₃)⁻ₙ (1);
Dⁿ⁺(R²OSO₃)⁻ₙ (2);
wherein R¹ is straight chain C₁₀-C₂₀ alkyl; R² is straight chain C₁₀-C₂₀ alkyl; n is an integer from 1 to 4, and D is selected from the group consisting of anti cancer drugs, anti bacterial drugs and anti fungal drugs or of a pharmaceutical composition comprising amphiphilic particulate sulfonate or sulfate powder of claim 7.

17. The method of claim 16, wherein administration is by perfusion, infusion or injection into a vein or artery.

18. The method of claim 16, wherein administration is to a solid tumour by infusion or injection into the peripheral circulation (i.e. intravenous injection), infusion or injection directly into the solid tumour, or by a bolus or by several boli.

19. The method of claim 18, wherein said solid tumour is a lung tumour, kidney tumour, liver tumour, pancreas tumour, breast tumour, or prostate tumour.

20. The composition of claim 2, wherein R¹ and R² is straight chain C₁₂-C₁₈ alkyl.
